(19) United States
(12) Patent Application Publication
(10) Pub. No.: US 2007/0093860 A1
(43) Pub. Date: Apr. 26, 2007

(54) SURGICAL METHOD AND CLAMPING APPARATUS FOR REPAIR OF A DEFECT IN A DURAL MEMBRANE OR A VASCULAR WALL, AND ANASTOMIC METHOD AND APPARATUS FOR A BODY LUMEN

(76) Inventors: Rob K. Rao, Hercules, CA (US); Mike Chen, Richmond, VA (US)

Correspondence Address: BLYNN L. SHIDELER;THE BLK LAW GROUP, 3500 BROKKTREE ROAD, SUITE 200, WEXFORD, PA, 15090, US

(21) Appl. No.: 11/533,623

(22) Filed: Sep. 20, 2006

Related U.S. Application Data
(60) Provisional application No. 60/718,926, filed on Sep. 20, 2005.

(51) Int. Cl. A61B 17/08

(52) U.S. Cl. 606/151

(57) ABSTRACT

A surgical method and apparatus of repairing a tear, cut or defect in the body tissue, specifically the dura or vascular wall, is disclosed. An inner plate is placed on the tissue's inner surface in a position completely overlapping the tissue defect. An outer plate is placed on the tissue's outer surface in a position completely overlapping the defect and aligned with the inner plate, whereby the inner and outer plates have perimeters larger than the perimeter of the defect. The inner plate is coupled to the outer plate such that the peripheral edges of the body tissue defect are securely clamped between the inner and outer plates to provide a watertight repair to the tissue defect. An associated clamping apparatus and insertion tool is disclosed. An anastomotic clamping device for a body lumen is also disclosed using opposed annular clamping plates.

### RELATED APPLICATIONS

The present application claims the benefit of U.S. Provisional Patent Application Ser. No. 60/718,926, filed Sep. 20, 2006 and entitled “Surgical Method and Clamping Apparatus for Repair of a Defect in a Dural Membrane or a Vascular Wall, and Anastomic Method and Apparatus for a Body Lumen”.

### BACKGROUND OF THE INVENTION

1. Field of the Invention

The present invention relates to repair of a defect in the dural membrane for spinal and cranial surgery, and to the repair of a defect in the vascular wall for vascular surgery, and to the anastomosis of a body lumen. More particularly the present invention relates to a surgical method and surgical clamping system for dural membrane repair, to a surgical method and surgical clamping system for vascular repair, and to a surgical method and surgical clamping system for anastomosis of a body lumen.

2. Background Information

The dura 10 (see FIG. 1), also called dural membrane and dura layer, is a layer of the membranous sac which covers the two parts of the central nervous system, the brain and spinal cord. A layer of fluid 12, termed cerebrospinal fluid, is present in the sub-arachnoid space between the dura and the structures of the central nervous system (i.e. the brain 14 or the spinal cord) and functions as a cushion as shown in FIG. 1. The other layers, the arachnoid layer 16 and pia layer 18, are very thin and structurally not significant for the purposes of the discussion in this application. The arachnoid layer 16 and the pia layer 18 are typically not specifically addressed in the repair of a rip, cut, rupture, tear, piercing or other defect in the dural membrane 10, in which a defect will generally also affect these structures. The term defect is used generically herein to reference all discontinuities in the membrane surface, including cuts, tears, naturally forming defects, rips, ruptures, piercing, or other break in the membrane surface.

The dura 10 is often damaged during surgery and requires repair so that cerebrospinal fluid 12 remains contained. A cerebrospinal fluid leak places the patient at substantial risk for meningitis (infection surrounding the brain), and generally causes a severe headache since the brain 14 sags without the supportive function of the fluid 12. The dura 10 is damaged purposefully (e.g. cut), on occasion, so that surgeons can access the underlying spinal cord or brain 14. Other times, the dura 10 is inadvertently injured during the course of spine surgery where access to the spinal cord is not required, i.e. removal of a herniated disc. The rate of inadvertent spinal fluid leaks due to dural membrane damage occurs in about 5% of open spinal procedures.

The numerous dural membrane repair methods can be generally categorized into: (a) those that re-approximate the edges of the defect (i.e. sutures or staples), (b) those that seal the defect with some type of glue, and (c) lastly, those that place a patch over the defect. Oftentimes, a combination of these strategies is used; however, significant drawbacks, which will be detailed further, are associated with each of these methods.

The first category of techniques, re-approximation of the edges, is the current method of choice and is represented in FIGS. 2a and 2b. Most commonly, fine suture 20, such as 4-0 silk available from US Surgical or 5-0 prolene available from Ethicon, is used to repair the dural defect. The suturing method is highly effective, but it is often not an option because of problems with either visualizing the dural membrane defect or with having enough room in the incision to manipulate the needle driver at the proper angle. Visualization of the defect 25 in the dura 10 can be difficult because the spine is often approached from the posterior (back) during surgery, as represented in FIG. 3, but the defect may occur in the anterior aspect (front) of the thecal sac. The spine 22, in FIG. 3, is being viewed behind and slightly off to the left of the patient. One analogy used to explain this relationship of elements is that the spine 22 is like a tunnel and the dural tube 10 is like a long worm going through it. The roof of the spinal canal is dissected away in FIG. 3 exposing the back and left side of the dural tube 10. Typical surgical exposure is rarely as good as shown in FIG. 3. The front and the sides of the dural tube 10 are essentially inaccessible to suturing instruments when the approach is from the posterior (back). Moreover, the back (side facing the surgeon) of the dural tube 10 is also extremely difficult to suture especially when the exposure is limited, as in microsurgery spine cases particularly when minimally invasive techniques such as endoscopes or tubes are used.

In an effort to provide surgeons with a tool that could compensate for the shortcomings of the suturing technique, titanium dural staplers, such as US Surgical's Auto Suture VCS™ disposable clip applier, were developed. These staplers possess the advantage of being able to work in tighter spaces; however, effective application is technically difficult for a number of reasons. One such reason is that these staplers are bulky and impede visualization of the affected area. Another frustrating problem is that the staples are difficult to place accurately, and to make matters worse, the staples have a known tendency to slip off.

The second major strategy for the repair of dural defects is the use of glues which are also referred to as tissue sealants. These glues are gelatinous masses that cover the defect, but do not actually glue the edges of the dura 10 together. Most of the approved biological sealants work through the basis of creating a fibrin mesh. When used by themselves, glues such as Tisseal™, are associated with significant drawbacks. One potential shortcoming is that tissue sealants require dry conditions to set; however, the spinal fluid leak is by definition a wet condition thus precluding use. Another concern is that the adhesive and tensile strength of the formed gels are lacking. Fluids tend to leak around the gelatinous mass, which is not firmly attached to the dura, or dissect through it. Because of these limitations, tissue sealants are commonly used as a supplement to other dural membrane closure techniques.

The third major tactic for repairing cerebrospinal fluids leaks is the use of a graft to patch over the defect. Several types of patches are available ranging from those harvested from the patient to those of the synthetic variety. The handling characteristics of these grafts vary widely and as such each type will be individually discussed.

Harvested grafts include those consisting of fat and muscle. If possible these patches are placed into the defect as a plug; otherwise, they are used like a blanket to cover the dural membrane defect, such as represented in FIG. 4. Sometimes the fat or muscle is secured to the dura 10 with stitches. Overall, these natural patches are effective and are used especially in cases where the spinal fluid leak is difficult to stop. The main drawbacks, however, are that significant additional tissue trauma is incurred with the act of harvesting, and that achieving a secure “plug” is not easy.

One alternative to fat and muscle grafts is bovine pericardium such sold under the brand name Duraguard™ by Synovis. In using animal tissues, the patient is spared the additional trauma of harvesting. However, since pericardium possesses no inherent stickiness to dura 10, it is a patch that must be sutured in water tight fashion into the defect. While it is often used to repair extremely large dural membrane defects for brain surgery, the need to suture the perimeter of the Duraguard™ patch to the free edges of dura 10 essentially precludes the use of this technique in the spine. Patches made of synthetic collagen matrices represent an additional option that is commonly employed. The difference between these patches, such as sold under the brand Duragen™ sold by Integra, and the bovine pericardium patches is that they possess some inherent stickiness to the dura 10 that allows the Duragen™ patches to be placed over the defect and secured without the use of sutures. This feature allows for more ready utility in the spine procedures. However, without sutures, the seal is tentative, and is usually reinforced with a tissue sealant. Even this combination of the patch and tissue sealant is far from secure. As with the previously described methods, patients often have an extended hospital stay, remaining flat in bed for 3 to 5 days, to allow for healing so that the dura 10 is sealed. This form of graft is particularly effective for fixing dural membrane leaks that are difficult to visualize. A competing patch type dural membrane repair product is manufactured by Codman.

By using a combination of current dural membrane repair techniques, most dural membrane defects can be fixed. The drawbacks either relate to technical difficulty, additional patient suffering and cost, or lack of certainty. It will be difficult to improve the dural repair methods that exist currently through improvements in the specifics of these techniques alone. Advancements in suture and staple technology will have to overcome the fact that a suture/staple line will always be more prone to leaks than a solid seal, and will also be more time consuming. Though a large amount of sealant technology research is being performed, there is considerable difficulty in finding glues that will attach to wet surfaces and remain biocompatible at the same time. Graft technology, such as Duragen™ brand grafts, will also require a substantial leap to overcome the lack of adherence to dural membrane edges. However, they will continue to serve a function particularly when the dural membrane defect cannot be easily visualized.

As spine surgery progresses more and more from traditional large open incisions to minimally invasive surgery, the limitations of current dural membrane closure techniques have become more apparent since the mainstay, the traditional suturing techniques, become even more difficult to perform If a new device could safely, effectively and rapidly close dural tears, then the current techniques could be readily supplanted as well as adding to the armentarium of tools for true minimally invasive procedures. In our opinion, there is a growing need for effective and efficient surgical methods and apparatus for the repair of defects in the dural membrane.

Similar to the dural tube, blood vessels also possess a lumen. Blood vessels in the body are of two types, arteries that carry blood from the heart to other organs and veins that carry blood from the body back to the heart and lungs so that re-oxygenation can occur. Blood in arteries is under high pressure, and as a result, arteries have a relatively thick wall which can be comparable to that of the dura. The diameter of arteries varies considerably from millimeters to about 3 centimeters (the aorta). The pressure in veins is low, and as a result, the walls are very thin.

Blood vessels are often injured from trauma or inadvertently during surgery. Repair of blood vessels is performed in the fields of trauma surgery, transplant surgery, neurosurgery, cardio-thoracic surgery, vascular surgery, orthopedic surgery, and general surgery. Failure to repair damaged blood vessels can lead to death by exsanguination, stroke, venous insufficiency, and loss of an organ or limb.

When blood vessels are damaged, surgeons most often will elect to sacrifice the vessel using methods such as suture ligation, vascular clips, and electrocautery. Removing the artery or vein from circulation is extremely effective, but a poor option if the damaged blood vessel has an important function. For example, grave consequences would occur if the aorta, the main artery of the body, was ligated.

Several techniques can be used to repair damaged blood vessels while preserving them at the same time. One common method is the application of a thrombin soaked sponge or a hemostatic gel to the bleeding vessel. These devices cause a clot to form and are very effective at stopping low pressure and low flow bleeding. The limitation of this method is that vigorous bleeding cannot be easily controlled. Numerous companies (Tisseal, Surgifoam, Surgicel, Avitene, Fibrillar, Flowseal) make commercial versions of this device.

Another hemostasis technique is the use of suture to close the defect in the blood vessel. The success of this method varies according to the surgical exposure, size and type of blood vessel. Large arteries and veins can be sutured under optimal conditions. However, placing these sutures is time consuming and often causes critical narrowing the vessels which could lead to inadequate circulation.

A third option is the use of electro-cautery techniques to close the defect. In this method a device such as the bipolar or electrosurgical pencil causes the tissue surrounding the defect to shrink and hopefully close the gap. Only very small defects with low flow bleeding can be treated with this technique and the risk of damaging the normal portions of the affected blood vessel is substantial.

One final alternative to closure of defects in blood vessels is the use of a patch. These patches can be natural (i.e. saphenous vein graft) or synthetic (Dacron® or polytetrafluroethylene (PTFE-Goretex®). Furthermore, they exists in different configurations such as a flat patch or in the form of a tube. Their use as a device to close vessel wall defects is limited for several reasons. First and foremost is the technical difficulty of sewing in these grafts particularly when time is of the essence and exposure is less than optimal as occurs in a emergency situations. Second, placement of these grafts necessitates a large surgical exposure which may not exist. Lastly, many of these grafts do not exhibit long term patency.

Taken together, the current methods for repair of damaged blood vessels possess limitations similar to those associated with existing methods of dural membrane repair. There is a need for effective and efficient surgical methods and apparatus for the repair of defects in vascular walls.

Neurosurgeons, cardiac surgeons, vascular, and transplant surgeons often need to reroute existing blood vessels to create what is known as a vascular bypass. Perhaps, the best known type of vascular bypass is the coronary artery bypass which is performed about 750,000 times per year in the United States. When an artery which brings blood to a part of the body is clogged the bypass is used to divert blood from non-critical areas. To create a bypass, a blood vessel must be anastomized (connected) to another one. The standard method to perform vascular anastomosis is by making a slit in the receiving vessel and then suturing the open lip of the donor vessel to this slit. The suture line is often reinforced with a hemostatic sponge and a sealant. Though this technique as depicted in the figure seems straightforward, it is in actuality very difficult because the blood vessels that surgeons anastomize are very small. As such, anastomosis is a difficult technique that is time consuming and very prone to failure. Failure occurs when the suture line leaks or when the graft does not remain as a viable pathway.

One new approach in the vascular anastomotic field is the development of the PAS-Port™ Proximal Anastomosis System, by Cardica, which is essentially a complicated stapler where the blood vessel is fed into the device and stapled to another vessel. Vascular anastomotic stapling works well in certain situations. However, the use of vascular staplers is limited by the bulkiness of the device and the inability to couple smaller vessels.

There is a need for effective and efficient surgical vascular anastomotic methods and apparatus. The anastomosis of body lumens is not limited to the vascular fields, but is used in other fields as well. The esophageal-gastro-intestinal tract has anastomotic applications that are well known, such as colon resections, gastric bypasses and the like. These other body lumen fields do not suffer all of the same drawbacks as found in the vascular area, for example the colon can readily accept staples for anastomosis and Ethicon Endo-surgical and U.S. Surgical have lines of surgical staplers to address this fields. Regardless, there remains a need for effective and efficient surgical anastomotic methods and apparatus for body lumen, not limited to the vascular areas.

### SUMMARY OF THE INVENTION

The concept behind the present invention addressing at least some of the above issues and relating to the repair of a defect in dural membrane or vascular wall is simply to trap the edges of the defect in the dura or vascular wall using two plates secured to one another. The concept behind the present invention relating to the vascular anastomosis addressing at least some of the above issues is to surround and clamp the edges of the opening in the graft receiving vascular wall using two annular plates secured to one another, with the bypass graft coupled to the outer annular plate. The concept for vascular anastomosis may be expanded for use with other body lumen anastomosis within the scope of the present invention.

A surgical method of and apparatus for repairing a defect in the dura according a non-limiting embodiment of the present invention includes placing an inner plate on an inner surface of the defect in the dura in a position overlapping the defect in the dura, whereby the inner plate has a perimeter in plan view larger than the perimeter in plan view of the defect in the dura. An outer plate is placed on an outer surface of the defect in the dura in a position completely overlapping the defect in the dura and aligned with the inner plate, whereby the outer plate has a perimeter in plan view larger than the perimeter in plan view of the defect in the dura. The inner plate is coupled to the outer plate such that the peripheral edges of the defect in the dura is securely clamped between the inner and outer plates to provide a watertight repair to the defect in the dura.

A surgical method of and apparatus for repairing a defect in a vascular wall according a non-limiting embodiment of the present invention includes placing an inner plate on an inner surface of the defect in the vascular wall in a position completely overlapping the defect in the vascular wall, whereby the inner plate has a perimeter in plan view larger than the perimeter in plan view of the defect in the vascular wall. An outer plate is placed on an outer surface of the defect in the vascular wall in a position completely overlapping the defect in the vascular wall and aligned with the inner plate, whereby the outer plate has a perimeter in plan view larger than the perimeter in plan view of the defect in the vascular wall. The inner plate is coupled to the outer plate such that the peripheral edges of the defect in the vascular wall is securely clamped between the inner and outer plates to provide a watertight repair to the defect in the vascular wall.

A surgical body lumen anastomotic method and apparatus, in particular vascular anastomosis, according a non-limiting embodiment of the present invention includes placing an inner annular plate on an inner surface of the graft receiving vascular wall in a position completely overlapping the bypass opening in the vascular wall. A bypass graft is coupled to an annular outer plate and the outer plate is placed on an outer surface of the graft receiving vascular wall in a position completely overlapping the bypass opening in the vascular wall and aligned with the annular inner plate. The inner plate is coupled to the outer plate such that the peripheral edges of the vascular wall around the bypass opening is securely clamped between the inner and outer plates to provide a watertight coupling in the vascular wall for the bypass graft attached to the outer annular plate.

These and other advantages of the present invention will be clarified from the attached figures wherein like reference numerals represent like elements throughout.

### BRIEF DESCRIPTION OF THE FIGURES

FIG. 1 is a schematic drawing of the different membranes covering the brain;

FIGS. 2a and 2b are schematic drawings depicting a dural membrane defect and closure of the defect with sutures, respectively;

FIG. 3 is a schematic anatomical representation of the spine from a posterior approach;

FIG. 4 is a schematic of a natural tissue graft used to close a dural tube defect to seal a cerebrospinal fluid leak;

FIG. 5 is a schematic view of the insertion step of a retracted inner plate of a dural membrane clamping apparatus according to one embodiment of the present invention through a dural defect to be sealed;

FIG. 6 is a schematic view of a deployment of the inner plate of FIG. 5;

FIG. 7 is a schematic view of the inner plate of FIG. 5 in a position adjacent the dural defect and an outer plate of the clamping apparatus according to one aspect of the present invention being moved into position;

FIG. 8 is a schematic view of the clamping apparatus of FIG. 7 in a final dural membrane repairing position;

FIG. 9 schematically illustrates interlocking surface ridges for the inner and outer plates of the clamping apparatus according to one embodiment of the present invention;

FIG. 10 is a perspective schematic view of an expanding inner plate and an integral locking stem coupling configuration for securing the inner and outer plates of the clamping apparatus according to one embodiment of the present invention;

FIG. 11 is a perspective view of an outer plate configured to couple with the inner plate and stem of FIG. 10;

FIGS. 12a-d are schematic views of an expanding inner plate and an integral locking stem coupling configuration for securing the inner and outer plates of the clamping apparatus according to another embodiment of the present invention;

FIG. 13 is a schematic view of an inner plate and an integral locking stem and separable handle coupling configuration for securing the inner and outer plates of the clamping apparatus according to another embodiment of the present invention;

FIG. 14 is a schematic view of an inner plate and an integral locking stem and separable handle coupling configuration for securing the inner and outer plates of the clamping apparatus according to another embodiment of the present invention;

FIG. 15 is a schematic view of a clamping apparatus and separable outer plate pusher configuration according to another embodiment of the present invention;

FIG. 16 is a schematic view of a clamping apparatus and separable outer plate pusher configuration with retracted inner and outer plates and outer sheath according to another embodiment of the present invention;

FIG. 17 is a schematic view of the clamping apparatus of FIG. 16 with the plates in a deployed position;

FIG. 18 is a schematic view of a vascular wall defect clamping mechanism according to one embodiment of the present invention;

FIGS. 19-20 are schematic views showing the deployment of an inner annular plate for a vascular anastomotic device according to one embodiment of the present invention;

FIGS. 21-22 are schematic views showing the attachment of a vascular bypass graft to an annular outer plate for attachment with the inner annular plate of FIGS. 19-20; and

FIG. 23 is a schematic view of the assembled vascular device of FIGS. 19-22.

FIG. 24 is a schematic perspective view of an inner plate and an integral locking stem configuration of the clamping apparatus according to another embodiment of the present invention;

FIG. 25 is a schematic perspective view of an inner plate and an integral locking stem configuration of the clamping apparatus according to another embodiment of the present invention;

FIG. 26 is a schematic perspective view of an outer plate of the clamping apparatus using the inner plates of FIGS. 24-25;

FIG. 27 is a schematic perspective view of an inner plate and an integral locking stem configuration of the clamping apparatus according to another embodiment of the present invention;

FIG. 28 is a schematic perspective view of an outer plate of the clamping apparatus using the inner plates of FIG. 27; and

FIG. 29 is a photograph of a prototype depicting one of the possible mechanisms for deployment of the inner plate.

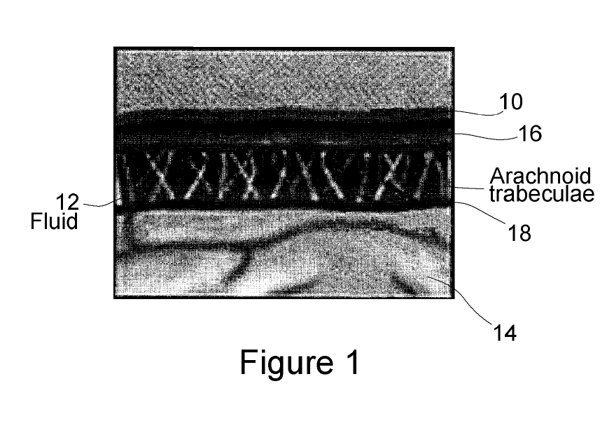

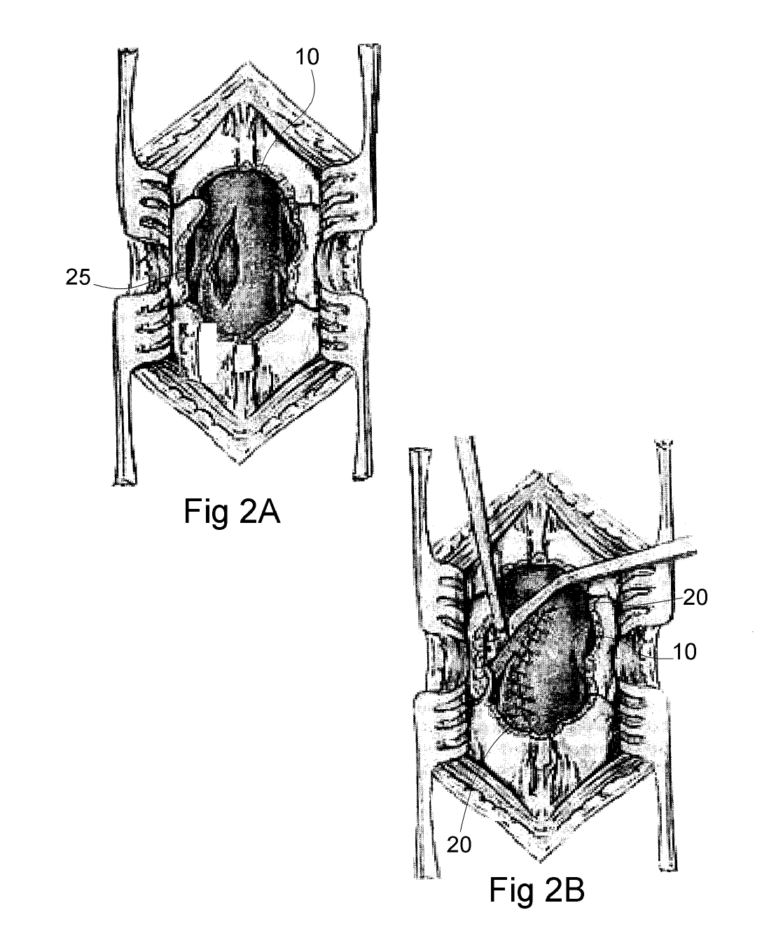

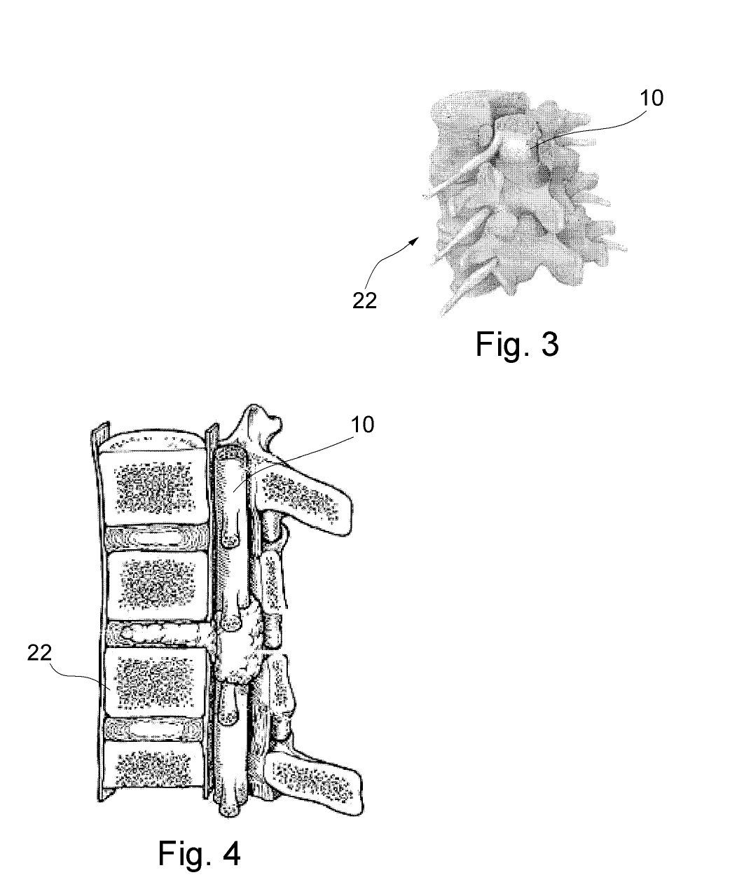

### DESCRIPTION OF THE PREFERRED EMBODIMENT

As noted above in the summary, the concept behind a dural defect surgical clamping apparatus 100 of the present invention is simply to trap the edges of the defect 25 in the dura 10 using two plates 110, 120 secured to one another. The components of the clamping apparatus 100 include an inner plate 110 that is positioned on the inside of the dura 10 (through the defect 25 to be sealed), an outer plate 120 opposed to the inner plate 110 and a coupler 130 to secure the two plates 110 and 120 together. Possible materials for the plates 110 and 120 include: poly-ethyl-ethyl-ketone (PEEK), high molecular weight poly-ethylene, silastic, titanium alloys, polypropelene, poly-glycolic acid, and poly-lactic acid. The latter two materials are bio-absorbable, more precisely bio-resorbable, and can be reinforced with carbon fiber. The term “biodegradable” refers to a biological mediated degradation process such as enzymatic and/or cellular processes. “Bioresorption” refers to a chemically mediated degradation process such as hydrolysis where the degradation products are then incorporated into normal metabolic pathways like the Krebs Cycle. “Bio-absorbable” technically also refers to a chemically mediated degradation, but the degradation products are generally excreted through one of the body's organ systems. All three terms are unfortunately used indiscriminately in both scientific and clinical literature and this has caused significant confusion. Within the meaning of this application “bio-absorbable” will be used in its broadest sense in the art and will therefore generically reference materials that are biodegradable, bio-resorbable or bio-absorbable in accordance with the above definitions.

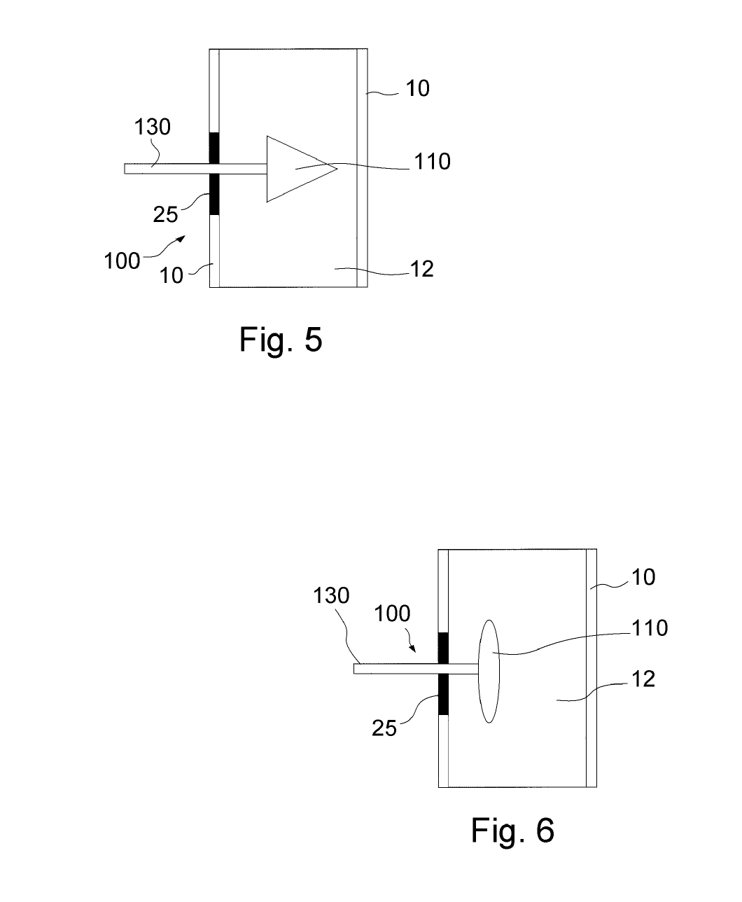

In one aspect of the present invention the inner plate 110 may be moved to a retracted insertion position to aid in placement beneath the dura 10. The inner plate 110 may also be a circular plate in plan view, although any shape of plate could be used. A circular shape for the interior plate 110 does provide symmetrical advantages. Depending on the size and shape of the defect as well as the presence of sensitive underlying neurological structures, the inner plate 110 can be inserted in the closed un-deployed position as shown in FIG. 5 or in the open fully deployed position, if possible. Following the insertion of the retracted un-deployed position of the inner plate 110, the retracted inner plate 110 can then open and fully deploy as the inner plate 110 as shown in FIG. 6. This deployment step also adds greater safety by allowing errant nerves, and other tissue, to be pushed out of the way during the opening of the inner plate 110, preventing the clamping of unwanted tissue between the two plates 110 and 120 when finally installed. These nerves are occasionally inadvertently trapped by staples or sutures in prior art methods.

The retraction and deployment mechanism may be through any appropriate mechanism. For example, the material forming the inner plate 110 may be flexed to the retracted position and held there against the elastic biasing force of the material forming the inner plate 110 by a separate holding member, such as sheath 112 of FIGS. 16-17, and when the holding member releases the contracted inner plate 110, the inner plate 110 returns to the fully deployed open position through the restoring force in the material itself. Another alternative is to have shape memory alloy strips (not shown), such as nitinol or titanium, incorporated into the inner plate 110, wherein in one possible configuration is formed as ribs like in an umbrella, and which are by default in a curved retracted position and then manipulated into the deployed position. In other words, the radial individual nitinol strips move from a tight “U” shape in the un-deployed retracted position to a straight shape in the deployed position. Other contracting and expanding devices may be used to contract and expand or deploy the inner plate 110.

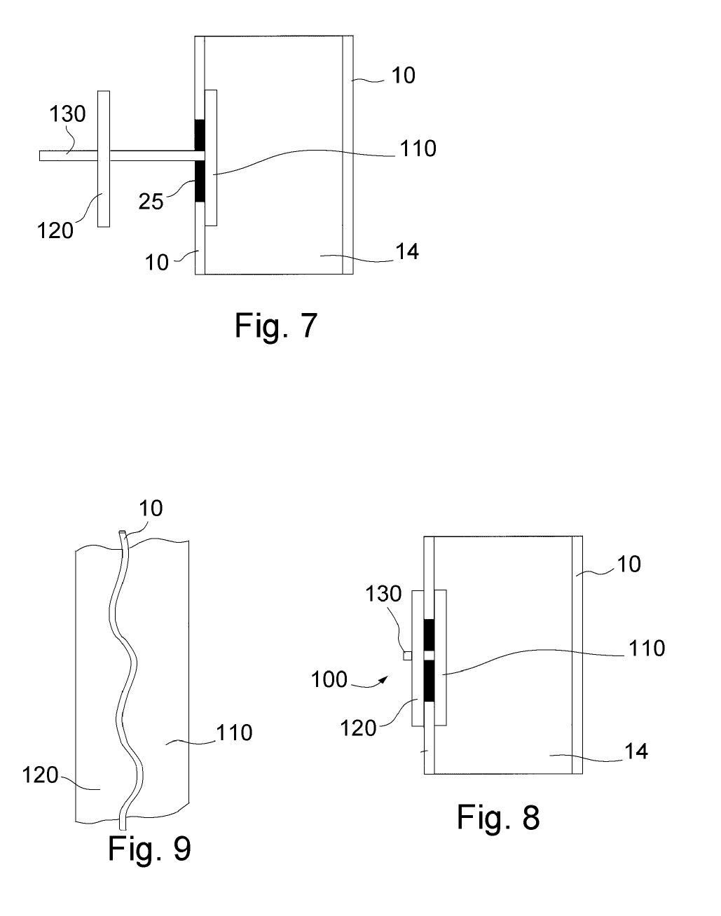

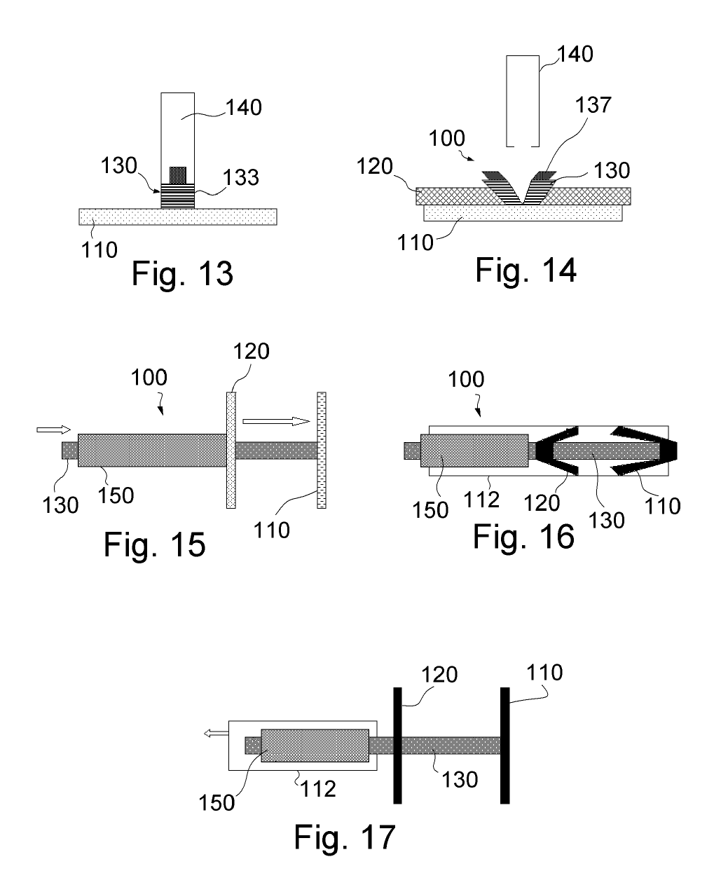

Following the positioning of the inner plate 110 in the interior of the dura 10, and deploying the inner plate 110 if it was inserted in a contracted position, the inner plate 110 is then brought into contact with the inner aspect of the dural defect as shown in FIG. 7. The matching outer plate 120 is then pushed down the coupler 130, or device stem, to lock with the inner plate 120 as shown in FIG. 8. It is important that the inner plate 110 and the outer plate 120 have a plane view larger than the periphery of the defect 25. A matching circular shape for the inner and outer plates 110 and 120 avoid alignment concerns. The circular profile for the inner and outer plates 110 and 120 allow the coupler 130 to include a threaded shaft portion 113 (shown in FIG. 13) engaging threads (not shown) on the outer plate 120, with the engaging threads allowing for sufficient clamping force to be created between the plates 110 and 120 in the final locked position. A locking washer or clip (not shown) could be used to prevent the outer plate 120 from backing off of the threaded shaft portion 133 (if threads are used on the coupler 130). The excess portion of the coupler 130, i.e. the portion of the shaft extending beyond the outer plate 130 (and any retaining clip) may be removed following installation. This installation clamping process traps the edges of the defect 25 in the dura between the plates 110 and 120, essentially placing a “manhole” cover over the defect 25. The faces of the plates 110 and 120 may further include meshing ridges and grooves as shown in FIG. 9 to further improve the seal around the defect 25. Further, a ratchet type stem could be used as one of many of the possible alternatives for the design of the coupler 130.

Other alternatives for the coupler configuration include use of adhesive material on the facing portions of the plates 110 and 120 to couple the inner and outer plates 110 and 120 together. The coupler 130 may include the use of a stem member as shown, primarily as a guide, and adhesives to provide the coupling compressive force between the plates.

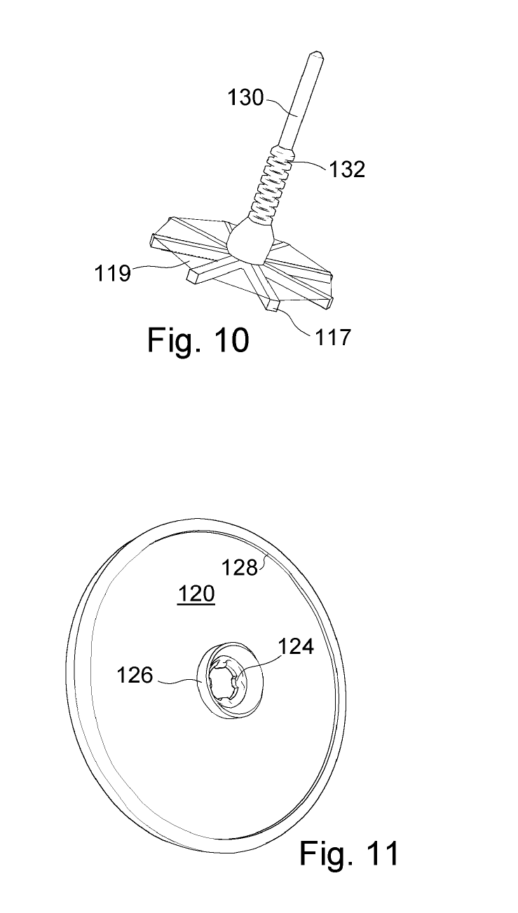

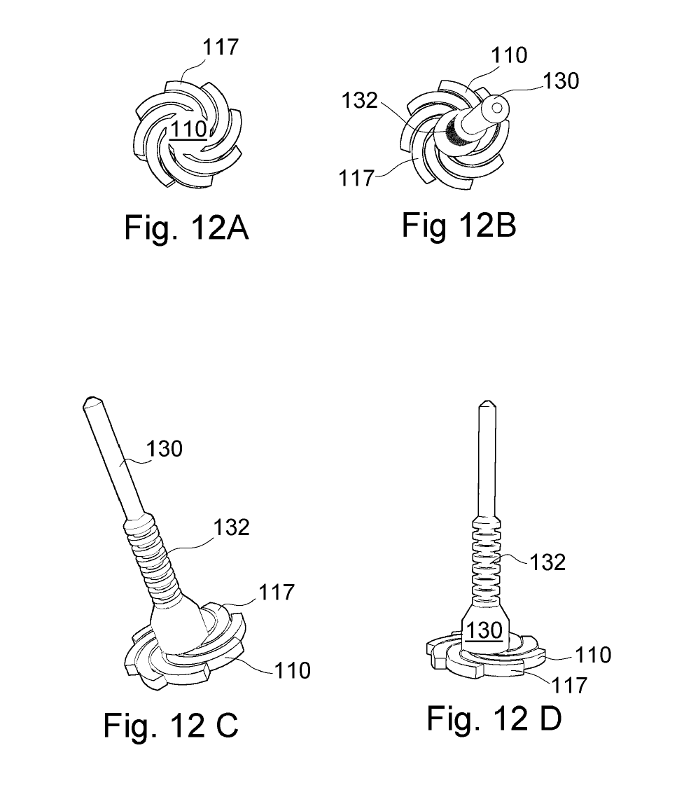

FIG. 10 is a perspective schematic view of an expanding inner plate 110 and an integral locking stem coupler 130 according to one embodiment of the present invention. The inner plate is formed of ribs 117 and flexible webbing 119 that easily allows for easy retraction to the contracted position shown in FIG. 5 above. The coupler 130 includes notches 132. FIG. 11 is a perspective view of an outer plate 120 configured to couple with the inner plate 110 and coupler 130 of FIG. 10. Specifically the plate includes flexible locking tabs 124 that engage within the notches 132. The tabs 124 are flexible enough to allow the plate 120 to be pushed down the stem of the coupler 130, with a beveled edge to allow the one-way movement. The plate may further include an alignment ring 126 on the inner side to assist in alignment with the inner plate 110. The outer plate 120 may be formed larger than the inner plate 110 with an outer retaining ring 128 as shown. FIGS. 12a-d are schematic views of an expanding inner plate 110 and an integral locking stem coupler 130 as described above in FIG. 10, except that the ribs 117 are formed in a coil configuration.

FIG. 13 is a schematic view of an inner plate 110 and an integral locking stem coupler 130 (with threaded portion 113) and separable handle 140 according to another embodiment of the present invention. This embodiment of the apparatus 100 a separable handle 140 allows for easy deployment of the inner plate 110, without separate trimming of excess stem portion. Any number of releasable connections between the separable handle 140 and the stem of the coupler 130 can be used. FIG. 14 is a schematic view of an inner plate 110 and an integral locking coupler 130 and separable handle 140 configuration according to another embodiment of the present invention. In this embodiment the coupler 130 is split and after the outer plate 120 is moved into position the handle 140 is removed and the split coupler 130 is spread apart as shown to lock the components together. A coupler handle 137 may be used to open the halves of the split coupler 130. This embodiment could be used with other coupling techniques such as threads.

FIG. 15 is a schematic view of a clamping apparatus 100 together with a separable outer plate pusher 150 according to another embodiment of the present invention. The outer plate pusher 150 is simply a mechanism to allow for remote attachment of the outer plate 120. The outer plate pusher 150 is separate from the outer plate 120 and will be removed, with excess stem of the coupler 130 if a separate handle 140 is not utilized, after installation. FIG. 16 is a schematic view of a clamping apparatus 100 with separable outer plate pusher 150, further including a plate holding sheath 112 as discussed above. In this configuration the inner plate 110 and the outer plate 120 are retractable. The retracted inner and outer plates 110 and 120 and outer sheath 112 according to this embodiment of the present invention allows for minimally invasive applications of the apparatus 100. FIG. 17 is a schematic view of the clamping apparatus 100 of FIG. 16 with the plates 110 and 120 in a deployed position, and it will be clear that the inner plate 110 will likely be deployed on the inside surface of the dural membrane 10.

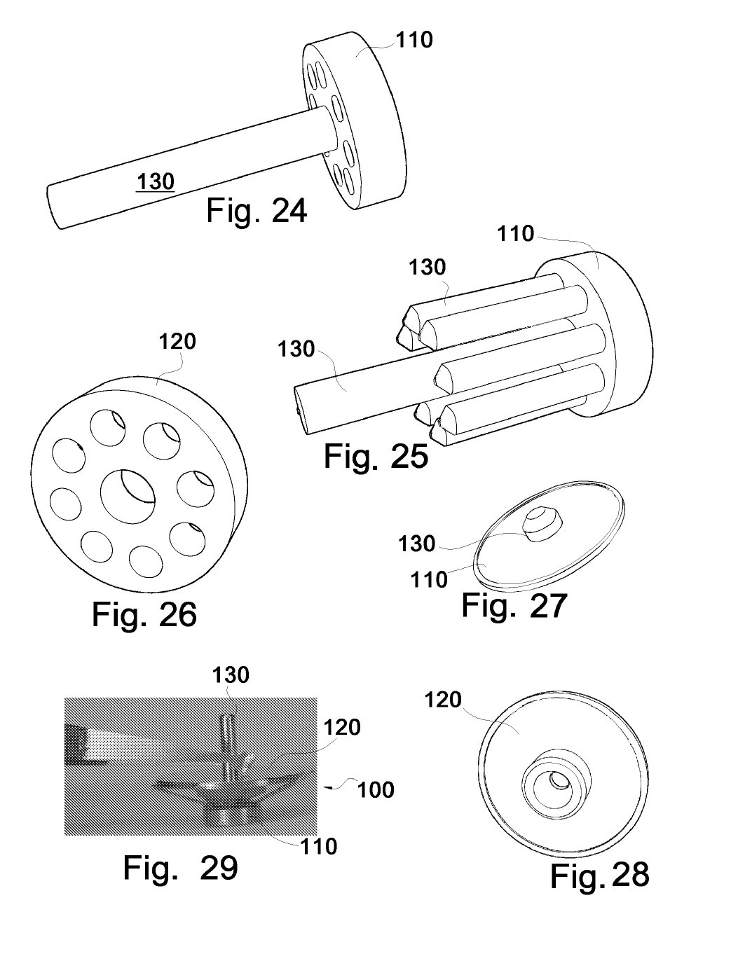

FIGS. 24-26 illustrate further embodiments of the apparatus 100 according to the invention. FIG. 24 is a schematic perspective view of an inner plate 110 and an integral locking stem 130, wherein the plate 110 includes one or more recesses therein. Where adhesive or the like is used to secure the plates 110 and 120, the recesses themselves will not affect dura defect sealing. FIG. 25 is a schematic perspective view of an inner plate 110 and an integral locking stem(s) 130 configuration in a plurality of locking stems are provided. FIG. 26 is a schematic perspective view of an outer plate 120 of the clamping apparatus 1000 that will correspond to using the inner plates 110 of FIGS. 24-25;

FIGS. 27-28 illustrate a further embodiment of the apparatus 100 according to the invention. FIG. 27 is a schematic perspective view of an inner plate 110 and an integral locking stem configuration 130 of the clamping apparatus 100 and FIG. 28 is a schematic perspective view of an outer plate 120 of the clamping apparatus 100 using the inner plates of FIG. 27.

FIG. 29 is a photograph of a prototype of the apparatus 100 according to the invention depicting one of the possible mechanisms for outward sweeping deployment of the inner plate 120 described above.

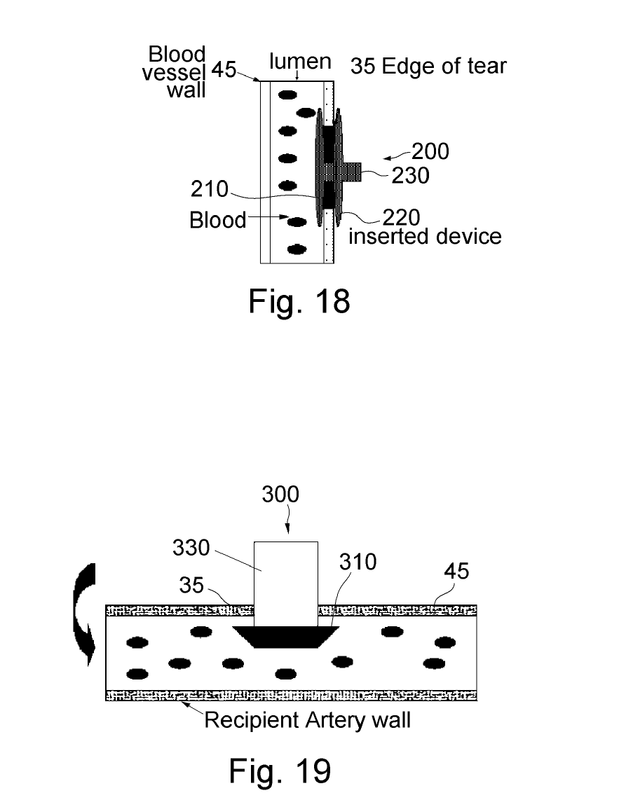

The present invention discloses a surgical apparatus 200 and associated method for repairing a defect 35 in a vascular wall 45 according a non-limiting embodiment of the present invention as shown in FIG. 18. This method includes placing an inner plate 210 on an inner surface of the defect 35 in the vascular wall 45 in a position completely overlapping the defect 35 in the vascular wall 45, whereby the inner plate 210 has a perimeter in plan view larger than the perimeter in plan view of the defect 35 in the vascular wall 45. An outer plate 220 is placed on an outer surface of the defect 35 in the vascular wall 45 in a position completely overlapping the defect 35 in the vascular wall 45 and aligned with the inner plate 45, whereby the outer plate 220 has a perimeter in plan view larger than the perimeter in plan view of the defect 35 in the vascular wall 45. The inner plate 210 is coupled to the outer plate 220 through a coupler 230 such that the peripheral edges of the defect 35 in the vascular wall 45 are securely clamped between the inner and outer plates 210 and 220 to provide a watertight repair to the defect 35 in the vascular wall 45.

The inner plate 210 may be formed in the manner discussed above in connection with the inner plate 110 and is analogous thereto. The outer plate 220 may be formed in the manner discussed above in connection with the outer plate 120 and is analogous thereto. Further, the coupler 230 may be formed in the manner discussed above in connection with the coupler 130 and is analogous thereto.

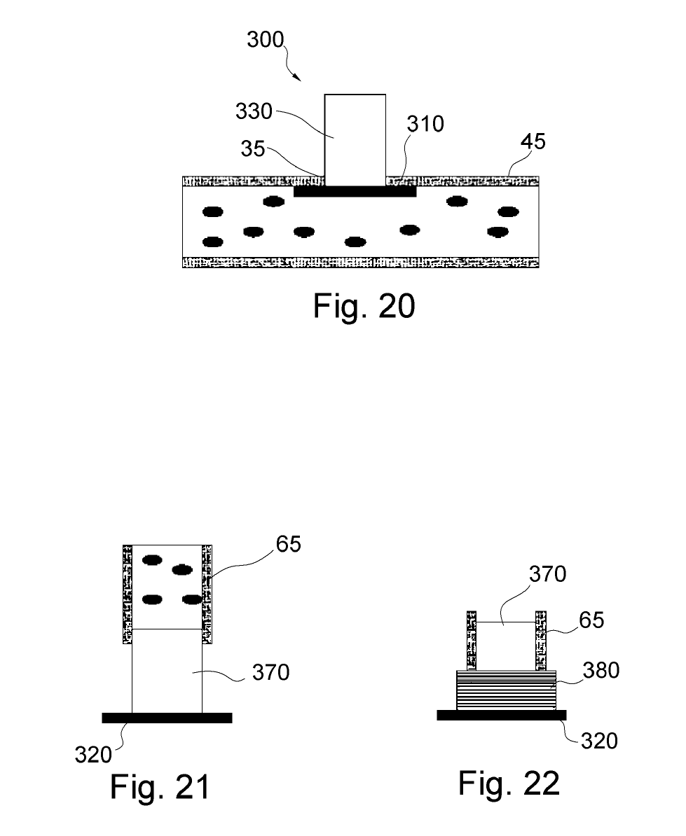

A surgical vascular anastomotic apparatus 300 according a non-limiting embodiment of the present invention includes placing an inner annular plate 310 on an inner surface of the graft receiving vascular wall 45 in a position completely overlapping the bypass opening 35 in the vascular wall. The bypass opening 35 is essentially a planned or inserted “defect” in the vascular wall 45 and thus uses the same reference numeral. The central opening in the annular plate 310 aligns with the bypass opening 35. FIGS. 19-20 are schematic views showing the deployment of the inner annular plate 310 for the vascular anastomotic device 300 according to one embodiment of the present invention. The inner annular plate 310 includes a hollow coupler 330.

A bypass graft 65 is coupled to an annular outer plate 320 such as shown in FIGS. 21 and 22. The outer plate 320 my include a hollow coupling stem 370 and matching locking ring collar 380 to secure the graft 65 there between, through friction or together with adhesives, or other coupling mechanisms that may be known in the art.

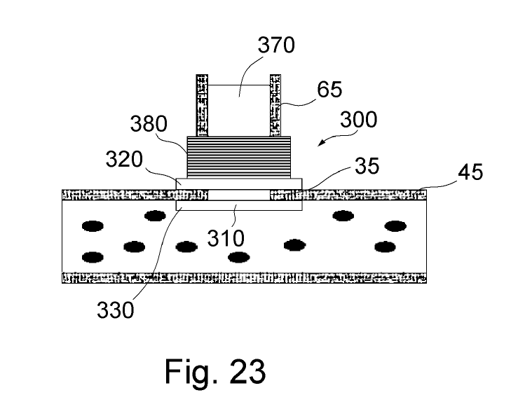

The annular outer plate 320 is placed on an outer surface of the graft receiving vascular wall 45 in a position completely overlapping the bypass opening 35 in the vascular wall 45 and aligned with the annular inner plate 310. The inner plate 310 is coupled to the outer plate 320 through coupler 330 such that the peripheral edges of the vascular wall 45 around the bypass opening 35 is securely clamped between the inner and outer plates 310 and 320 to provide a watertight coupling (other than through the bypass opening 35 and graft 65) in the vascular wall 45 for the bypass graft 65 attached to the outer annular plate 320. FIG. 23 is a schematic view of the assembled vascular anastomotic assembly 300.

The annular inner plate 310 may be formed in the manner discussed above in connection with the inner plate 110 and is analogous thereto, except for the central hole in the plate 310 allowing flow through the bypass graft 65. The outer plate 320 may be formed in the manner discussed above in connection with the outer plate 120 and is analogous thereto, except for (1) the central hole in the plate 320 allowing flow through the bypass graft 65 and (2) a mechanism to couple the graft 65 thereto. Further, the coupler 330 may be formed in the manner discussed above in connection with the coupler 130 and is analogous thereto, although coupler 330 must allow flow through the bypass graft 65.

It should be apparent that there are many variations to the present invention that can be found within the spirit and scope of the present invention. The key aspects of the surgical method and surgical clamping apparatus of repairing a defect in the dura is (a) placing an inner plate having a perimeter, or plan view, larger than the perimeter of the defect on an inner surface of the defect in the dura in a position completely overlapping the defect in the dura, (b) placing an outer plate having a perimeter, or plan view, larger than the perimeter of the defect on an outer surface of the defect in the dura in a position completely overlapping the defect in the dura and aligned with the inner plate, and (c) coupling the inner plate to the outer plate such that the peripheral edges of the defect in the defect in the dura is securely clamped between the plates to provide a watertight repair to the defect in the dura. Similar methods and apparatus are described for the repair of a defect in the vascular wall and for anastomosis of a body lumen, in particular a vascular anastomosis. The surgical method and surgical clamping apparatus is intended to be defined by the appended claims and equivalents thereto.

What is claimed is:

1. A surgical method of repairing a defect in the dura or blood vessel comprising the steps of:
(a) placing an inner plate on an inner surface of the defect in the dura or blood vessel in a position completely overlapping the defect in the dura or blood vessel, whereby the inner plate has a perimeter in plan view larger than the perimeter in plan view of the defect in the dura or blood vessel;
(b) placing an outer plate on an outer surface of the defect in the dura or blood vessel in a position completely overlapping the defect in the dura or blood vessel and aligned with the inner plate, whereby the outer plate has a perimeter in plan view larger than the perimeter in plan view of the defect in the dura or blood vessel; and
(c) coupling the inner plate to the outer plate such that the peripheral edges of the defect in the dura or blood vessel is securely clamped between the inner and outer plates to provide a watertight repair to the defect in the dura or blood vessel.

2. The method of claim 1 wherein the inner and outer plates are circular in plan view.

3. The method of claim 1 wherein the defect is in a blood vessel and the clamping apparatus is configured for use on a defect in a blood vessel.

4. The method of claim 1 wherein the coupling of the inner plate to the outer plate uses a guide stem extending from the inner plate to the outer plate.

5. The method of claim 4 further including the step of trimming an excess portion of the guide stem after the outer plate is coupled to the inner plate.

6. The method of claim 4 wherein the guide stem includes at least one of threads or notches that engage at least one of threads, notches or retaining tabs on the outer plate.

7. The method of claim 1 wherein the plates are formed of bio-absorbable material.

8. The method of claim 1 wherein the step of placing an inner plate on an inner surface of the defect comprises the steps of inserting the inner plate in a retracted position through the defect and then opening the inner plate to a fully deployed position.

9. The method of claim 8 wherein the opening of the inner plate is in a direction tending to move tissue away from the area of the dura having the defect.

10. The method of claim 1 wherein the dura with the defect is the dural tube within the spinal column.

11. A surgical clamping apparatus for repairing a defect in the dura or blood vessel, said dura clamping apparatus comprising:
(a) an inner plate configured to be placed on an inner surface of the defect in the dura or blood vessel in a position completely overlapping the defect in the dura or blood vessel, wherein the inner plate has a perimeter in plan view larger than the perimeter in plan view of the defect in the dura or blood vessel;
(b) an outer plate configured to be placed on an outer surface of the defect in the dura or blood vessel in a position completely overlapping the defect in the dura or blood vessel and aligned with the inner plate, wherein the outer plate has a perimeter in plan view larger than the perimeter in plan view of the defect in the dura or blood vessel; and
(c) a coupling member for coupling the inner plate to the outer plate such that the peripheral edges of the defect in the dura or blood vessel is securely clamped between the inner and outer plates to provide a watertight repair to the defect in the dura or blood vessel.

12. The surgical dura clamping apparatus of claim 11 wherein the inner and outer plates are circular in plan view.

13. The surgical dura clamping apparatus of claim 11 wherein the inner and outer plates are circular in plan view and the outer plate as a larger diameter than the inner plate.

14. The surgical dura clamping apparatus of claim 11 wherein the coupling member includes a guide stem extending from the inner plate to the outer plate.

15. The surgical dura clamping apparatus of claim 14 wherein the coupling member includes an adhesive.

16. The surgical dura clamping apparatus of claim 14 wherein the guide stem is a notched at a position generally adjacent the inner plate with the notches engaging retaining tabs on the outer plate.

17. The surgical dura clamping apparatus of claim 11 wherein the plates are formed of bio-absorbable material.

18. The surgical dura clamping apparatus of claim 11 wherein the inner plate is moveable at least between a retracted position for insertion of the inner plate through the defect and an open fully deployed position.

19. The surgical dura clamping apparatus of claim 18 wherein the direction of movement of the inner plate from the retracted to the open position is in a direction tending to sweep tissue away from the dura or blood vessel defect.

20. The surgical dura clamping apparatus of claim 11 wherein the outer plate can be retracted and deployed.
